(19) United States
(12) Patent Application Publication
(10) Pub. No.: US 2018/0268935 A1
(43) Pub. Date: Sep. 20, 2018

(54) Non intrusive, non invasive, plug-and-forget, contextually aware, dark data processing, care companion platform for care receiver management by care providers

(71) Applicant: magpie 360 Inc.A, Andover, MA (US)

(72) Inventors: Ramesh Tumkur, Westford, MA (US); Kannan Ramasamy, Andover, MA (US)

(21) Appl. No.: 15/067,422

(22) Filed: Mar. 11, 2016

Related U.S. Application Data
(60) Provisional application No. 62/177,637, filed on Mar. 20, 2015.

(51) Int. Cl. G16H 40/63; G16H 10/60; G16H 50/20

(52) U.S. Cl. CPC G16H 40/63; G16H 50/20; G16H 10/60

(57) ABSTRACT

The human body continually generates analog data signals depicting its real time state and well-being. Some of this data (“dark data”) goes unnoticed. The disclosed care companion platform provides a computing system for continually processing dark data. It seamlessly blends into the care receiver environment over a plug-and-forget process, becomes contextually aware of the surroundings, lock to a specific care receiver to effectively safeguard the care receiver's health and well-being. It enables the care givers to continually ascertain overall quality of care, comfort and security of the care receiver.

### RELATED APPLICATIONS

This application claims priority from U.S. Provisional Patent Application Ser. No. 62/177,637 filed on Mar. 20, 2015.

### BACKGROUND OF THE INVENTION

Care management plays a vital role in planning, evaluation, seamless health assessment and care intervention for improvements in comfort, life expectancy and overall health of the care receivers. It needs to be coordinated and managed by the care provider.

Effective care management and monitoring calls for increased effort and greater demand on the care provider as the care receiver transitions from the care providers' work site to a remote site such as the transport to and from the care providers' site, care receivers' home, assisted living, mental health institutions, and hospice centers. Care providers need to consider the privacy and independence of the care receiver, which includes data privacy and data security, when scoping the effort.

The use of specialized care professionals adds to the overall responsibilities, service overheads, risk management and cost overheads on the care provider. The goal is to provide care providers and care givers the tools to seamlessly co-ordinate and efficiently manage the care receiver while respecting and protecting the privacy and independence of the care receiver.

### SUMMARY

The human body continually generates analog data signals depicting its real time state and well-being. Some of this data (“dark data”) goes unnoticed. “Care givers” then miss signals, and therefore lose opportunities to intervene. This makes health care more expensive and lowers the quality of care.

Our invention (“care companion platform”) provides a computing system for continually processing dark data. It is intended to seamlessly blend into the care receiver environment over a plug-and-forget process, become contextually aware of the surrounding, lock to a specific care receiver to effectively safeguard the care receiver's health and well-being.

It enables the “care givers” to “continually” ascertain overall quality of care, comfort and security of the care receiver. At the same time it respects and protects the privacy and independence of the care receiver and enables the care receiver to know his current state and well-being.

It provides security and comfort to the “care receiver”, alleviates round-the-clock functional burden on the “care giver” and eases the process and business overhead on the “care provider”.

### BRIEF DESCRIPTION OF THE DRAWINGS

FIG. 1 illustrates the care companion platform for processing dark data.

FIG. 2 illustrates the care companion architecture blocks.

FIG. 3 illustrates the care zone.

FIG. 4 illustrates an example of care receiver monitoring.

FIG. 5 illustrates the weighted metric across the care platform.

### DETAILED DESCRIPTION

A “Care Companion Platform” for the care provider and care giver seamlessly managing the care receiver is disclosed.

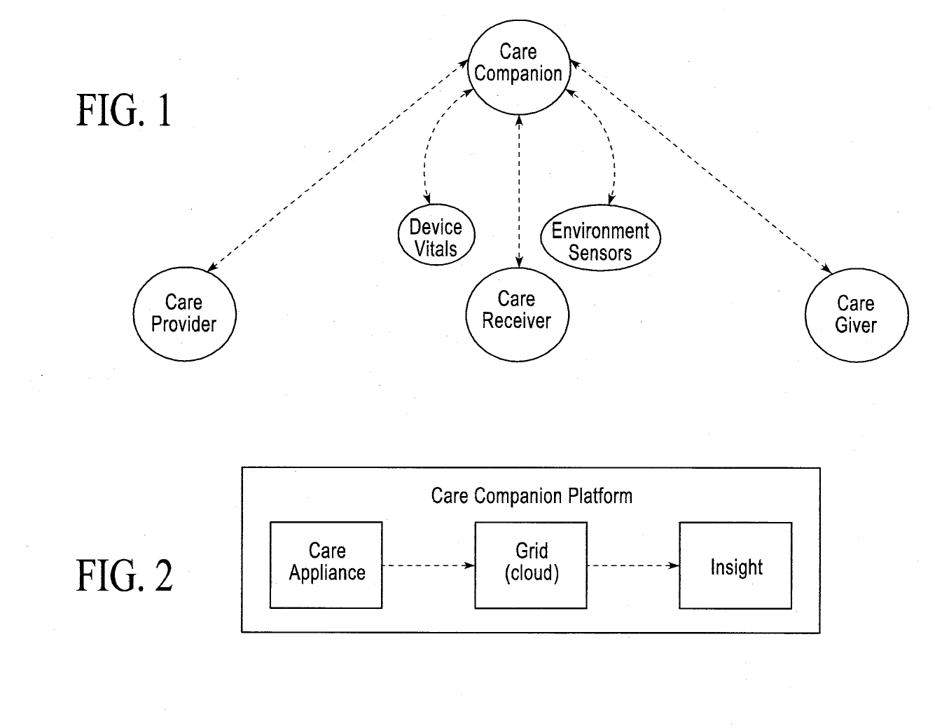

The higher-level embodiment that encompasses all other embodiment is the “care companion” illustrated in FIG. 1 that connects the “care provider” and “care giver” with the “care receiver”, The care companion provides the care receivers a way to know their current state and well-being.

The lower level embodiments illustrated in FIG. 2, are listed below.

1. A non-intrusive plug-and-forget wall mounted “care appliance” that contextually and “algorithmically” locks to the care receiver and the ecosystem for vitals and environmental awareness. Non-intrusively locking to the care receiver and thus staying contextually close to the care receiver until unlocked or retired from service enables the “care companion” to be a guardian without the need and awareness of the care receiver.

It is seamless and dynamic from the care receiver perspective. The “care appliance” also includes provision of a zoned perimeter and state of the care receiver environment pertinent to the remote care provider to continually ascertain the safety and well-being of the care receiver.

2. A “grid” for the “companion” events, flows and processes that makes contextual locking possible. The grid extends the ability of provider to monitor receiver without intrusion or interference. At the same time it effectively safeguards the health, privacy and independence of the care receiver.

3. “Insight” locks to the “grid” to provide defined actionable alerting, notification, dashboards and incident handling capabilities. It enables the authorized remote provider service to extrapolate the care receiver's state for further mapping and refinements. It also enables provider interaction, reports, glimpse of the care receiver state and conditions, notification, diagnostics, acknowledgements, closures and further interactions.

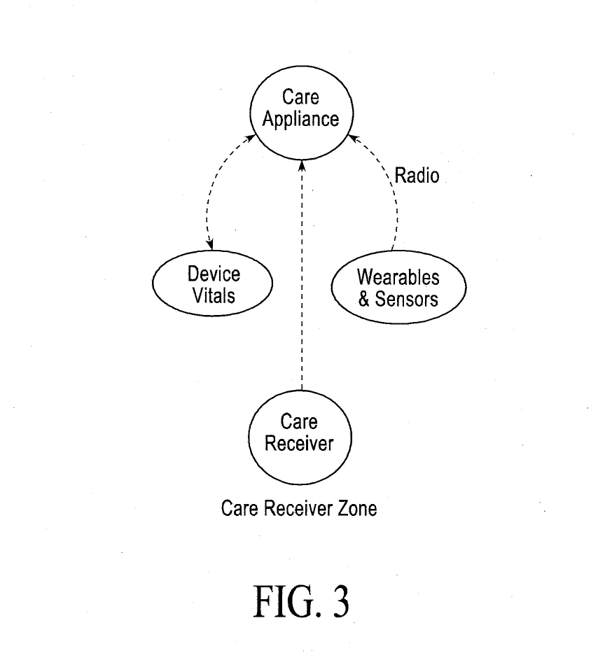

The system and methods that encompass the care receiver platform are further described below. Sensors bound at the care receiver as illustrated in FIG. 3, provide the contextual, situational and care receiver awareness forming the foundation for algorithmically establishing the relationship with the companion. Wearables, sensors and device vitals are recorded over radio at various states of transitions without care receiver awareness by way of a sensor engine at the companion appliance.

The radio packets emanating from the care receiver tag, sensor and vital monitors are extrapolated, normalized and transported upstream continually without any intervention by the “receiver zone”. The “receiver zone” includes the sensors, vital monitors and the “care appliance”.

The transport engine moves the packets upstream over secure and encrypted session to the care cloud's grid where correlation, normalization, analytics are applied as part of the service framework. The logic engine at the insight module provides the intelligence and contextual awareness for extrapolation into defined actionable alerts, reports, incidents and closed loop resolution through care intervention.

Care Appliance—Watching the Watcher: A weighted approach

From the point of view of care appliance, its security, reachability and availability round the clock is very critical to care companion platform. A failure of the care appliance and its communication link or the failure of the medical sensors including their battery upkeep will prevent vital measurement and thus impact the care receiver. This is crucial to point of care and thus needs to be monitored and managed proactively from remote in a heuristic process.

Thus this care platform has one of its kind “watching the watcher” feature of ascertaining that the care appliance, the communication link and the medical sensors are in always active state and nothing is lost in translation in safeguarding the care receiver. It does that with specific responses to potential failure modes:
When the device fails care platform automatically becomes aware of the situation.
When the batteries go down or the sensor is lost care platform automatically detects the issue.
When the intermediate network fails or when the internal processes dies or when hardware component fails, care platform gets notified.

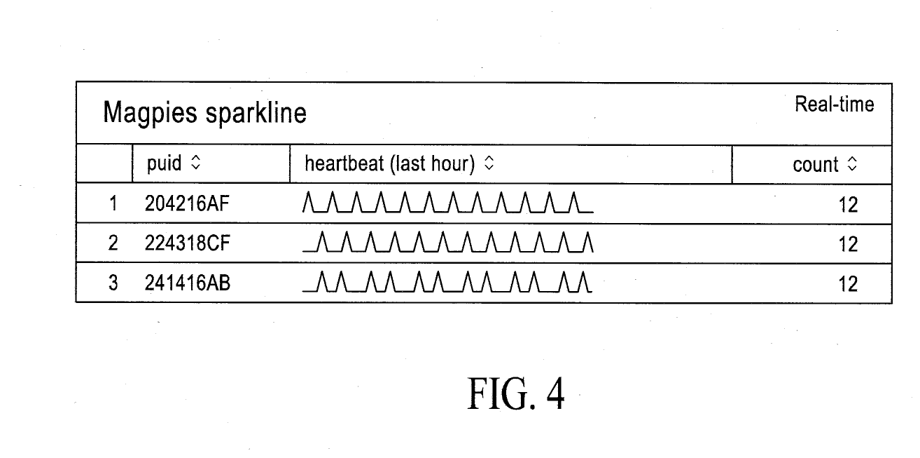

In all the above case the process involves computing the health of the device, connectivity to the sensor, reachability checks, battery duration and remaining charge, kernel parameters, security checks, ascertaining the links to the grid and finally a computed weighted value. The “device beat pulse” on the dashboard visual in FIG. 4 along with alerts give the care provider a complete sense of security of the eco system.

The pulse is weighted and produced at a fixed time interval so the environment-check is in place to ascertain that care-receiver and thus care zone is constantly and proactively monitored and issues ascertained pre-hand through an automated machine learning based loop.

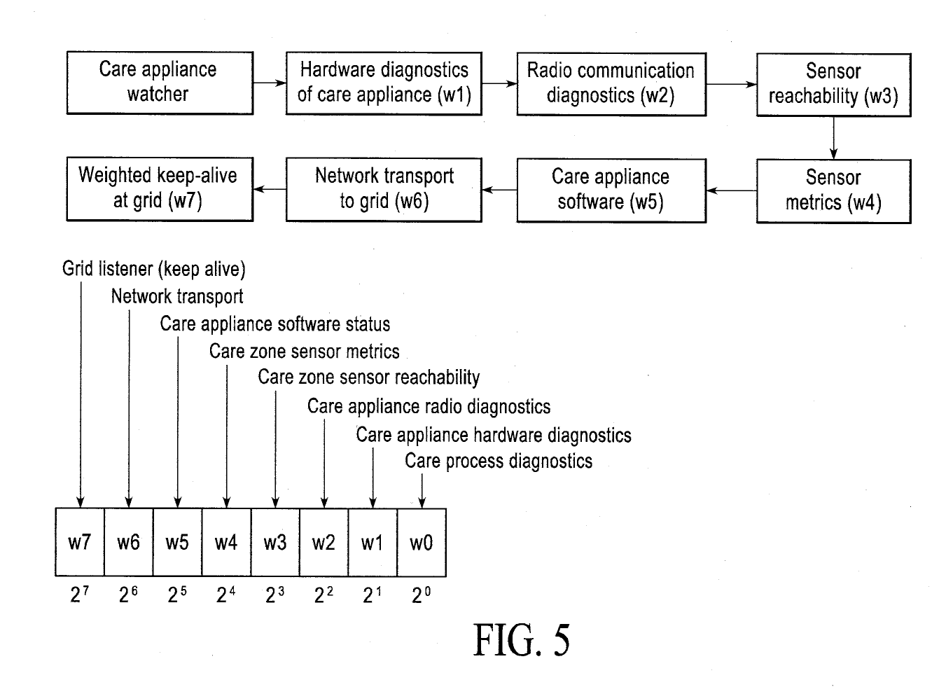

On a visual scale, the weighted value of “1” implies the entire eco-system is healthy and the care-receiver is being safe-guarded with “keep-alive” variable pointing to “1” in the spark-line. At any interval of time if the keep-alive value is “0” it implies care is being affected due to issues. The values are thus Boolean with keep-alive of “1” depicting “healthy-status” and a “0” for “impact-status”. The process is tied to a configurable time-ticker with each pulse arriving over a fixed duration. The non-arrival of the keep-alive within the configured time-tick would imply a impact-status allowing for further diagnostic trigger to identify the source. FIG. 4 references one such time-ticker attribute over “5” minute time interval where a weighted-1 shows up every 5 minute for a net count of 12 for an hour depicting “normalcy across the board”. The spark-line gives a quick visual indication of the health-status across each care-receiver virtual center. FIG. 5 depicts the weighted process.

What is claimed is:

1. A round the clock, virtual care center giving contextually aware care companion process that provides the quality of care experience of a care giver safeguarding the health and well-being of care receiver within the zone perimeter comprising:
(a) a wall mounted, plug-and-forget companion device that processes dark data from the human ecosystem;
(b) a plurality of sensors in the zoned perimeter making up the care receiver ecosystem locked to the companion device;
(c) seamless association and locking of the care-receiver representation sensor to the companion resource;
(d) a plurality of sensors locked to the companion representing the vital resources;
(e) a plurality of medical devices locked to the companion representing the health vitals;
(f) an intelligent transportation engine;
(g) a normalization, correlation and contextual engine part of backend platform in the cloud;
(h) an anomaly engine in the platform for alerts and ticketing;
(i) dashboards and user interfaces for feedback loop to the care-provider and care-receiver apps;
(j) mobile user interfaces for a real-time awareness of the care-giver state and well-being;
(k) providing continuous data and historical data over charts, tables and drill downs;
(l) mobile user-interfaces for alert tracking, incident handling, notifications and timelines;
(m) care provider and care receiver interaction over a user interface;
(n) notification policies, escalation policies, on-call handling, situational awareness & timelines; and
(o) watching the watcher feature with emphasis on the care receiver and care receiver environment for proactive monitoring of the environment, care appliance and medical sensors covering environment issues impacting the health and well-being of a care receiver through an automated machine learning process.
